(19) United States
(12) Patent Application Publication
(10) Pub. No.: US 2011/0203598 A1
(43) Pub. Date: Aug. 25, 2011

(54) NASAL DEVICES INCLUDING LAYERED NASAL DEVICES AND DELAYED RESISTANCE ADAPTERS FOR USE WITH NASAL DEVICES

(76) Inventors: Michael L. Favet, San Jose, CA (US); Arthur Ferdinand, San Jose, CA (US); Danny Yu-Youh Lai, San Jose, CA (US)

(21) Appl. No.: 13/035,524

(22) Filed: Feb. 25, 2011

Related U.S. Application Data
(63) Continuation-in-part of application No. 11/759,916, filed on Jun. 7, 2007.
Application No. 11/759,916 is a continuation-in-part of application No. 12/329,895, filed on Dec. 8, 2008.
(60) Provisional application No. 61/308,019, filed on Feb. 25, 2010.
(60) Provisional application No. 60/905,850, filed on Mar. 7, 2007.
(60) Provisional application No. 60/859,715, filed on Nov. 16, 2006.
(60) Provisional application No. 60/811,814, filed on Jun. 7, 2006.
(60) Provisional application No. 61/012,016, filed on Dec. 6, 2007.

(51) Int. Cl. A61F 5/56

(52) U.S. Cl. 128/848

(57) ABSTRACT

Described herein are layered nasal devices including layered nasal devices having one or more stiffening members supporting the holdfast region of the nasal device. The stiffening member may be a stress-distributing member or a separate stress-distributing element or member may be included. In some variations the layered nasal device includes a stress distributing element to help prevent wrinkling, de-laminating, buckling, or otherwise disrupting the shape and/or activity of the nasal device. Also described herein are delayed resistance adapters that may be used with a nasal devices that inhibit exhalation more than inhalation (including, but not limited to the adhesive nasal devices described herein). A delayed resistance adapter may be activated to suspend or bypass the increased expiratory resistance of the nasal device. Suspending the increased expiratory resistance may allow the user to allow a user to acclimate to the use of the nasal device.

### CROSS REFERENCE TO RELATED APPLICATIONS

This patent application claims priority to U.S. Provisional Patent Application No. 61/308,019, filed on Feb. 25, 2010, and titled “NASAL DEVICES INCLUDING LAYERED NASAL DEVICES AND DELAYED RESISTANCE ADAPTERS FOR USE WITH NASAL DEVICES”. This application is herein incorporated by reference in its entirety.

This patent application also claims priority as a continuation-in-part of U.S. patent application Ser. No. 11/759,916, filed on Jun. 7, 2007, titled “LAYERED NASAL DEVICES,” which claims priority to U.S. Provisional Patent Application Nos.: 60/905,850, filed on Mar. 7, 2007, titled “NASAL DEVICES”; 60/859,715, titled on Nov. 16, 2006, titled “NASAL DEVICES”; and 60/811,814, tiled on Jun. 7, 2006, titled “RESPIRATORY DEVICES”. Each of these patent applications is herein incorporated by reference in its entirety.

This patent application also claims priority as a continuation-in-part to U.S. patent application Ser. No. 12/329,895, filed on Dec. 8, 2008, titled “DELAYED RESISTANCE NASAL DEVICES AND METHODS OF USE,” which claims priority to U.S. Provisional Patent Application No. 61/012,016, filed on Dec. 6, 2007, titled “DELAYED RESISTANCE NASAL DEVICES AND METHODS OF USE”. Each of these patent applications is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD OF THE INVENTION

The devices and methods described herein include layered nasal devices and adapters configured to couple to a nasal device to enhance comfort by delaying or ramping the increased resistance to exhalation and/or inhalation.

### BACKGROUND OF THE INVENTION

Nasal respiratory devices have been well-described in the following patents and patent applications, each of which was previously incorporated in its entirety: U.S. patent application Ser. No. 11/805,496, filed on May 22, 2007, titled “NASAL RESPIRATORY DEVICES;” U.S. patent application Ser. No. 11/759,916, filed on Jun. 7, 2007, titled “LAYERED NASAL DEVICES;” U.S. patent application Ser. No. 11/298,339, filed on Dec. 8, 2005, titled “RESPIRATORY DEVICES,” U.S. patent application Ser. No. 11/298,362, filed on Dec. 8, 2005, titled “METHODS OF TREATING RESPIRATORY DISORDERS;” U.S. patent application Ser. No. 11/298,640, filed on Dec. 8, 2005, titled “NASAL RESPIRATORY DEVICES;” U.S. patent application Ser. No. 12/141,875, filed on Jun. 18, 2008, titled “ADHESIVE NASAL RESPIRATORY DEVICES,” U.S. patent application Ser. No. 11/811,401, filed on Jun. 7, 2007, titled “NASAL RESPIRATORY DEVICES FOR POSITIVE END-EXPIRATORY PRESSURE;” U.S. patent application Ser. No. 11/941,915, filed on Nov. 16, 2007, titled “ADJUSTABLE NASAL DEVICES;” U.S. patent application Ser. No. 11/941,913, filed on Nov. 16, 2007, titled “NASAL DEVICE APPLICATORS;” U.S. patent application Ser. No. 11/811,339, filed on Jun. 7, 2007, titled “NASAL DEVICES;” U.S. patent application Ser. No. 12/044,868, filed on Mar. 7, 2007, titled “RESPIRATORY SENSOR ADAPTERS FOR NASAL DEVICES;” U.S. patent application Ser. No. 12/369,681, filed on Feb. 11, 2009, titled “NASAL DEVICES;” U.S. patent application Ser. No. 12/364,264, filed on Feb. 2, 2009, titled “CPAP INTERFACE AND BACKUP DEVICES;” U.S. patent application Ser. No. 12/329,271, filed on Dec. 5, 2008, titled “PACKAGING AND DISPENSING NASAL DEVICES;” U.S. patent application Ser. No. 12/329,895, filed on Dec. 8, 2008, titled “DELAYED RESISTANCE NASAL DEVICES AND METHODS OF USE;” U.S. patent application Ser. No. 12/405,837, filed on Mar. 17, 2009, titled “NASAL DEVICES WITH NOISE-REDUCTION AND METHODS OF USE;” U.S. patent application Ser. No. 12/485,750, filed on Jun. 16, 2009, titled “ADJUSTABLE RESISTANCE NASAL DEVICES;”; international Patent Application No. PCT/US2009/056948, filed on Sep. 15, 2009, titled “NASAL DEVICES, SYSTEMS AND METHODS;” and U.S. Patent Application No, 61/258,865, filed on Nov. 6, 2009, titled “NASAL DEVICES HAVING A SAFE FAILURE MODE”

In general, these nasal respiratory devices are configured to inhibit exhalation more than inhalation in a sleeping patient. The resistance to exhalation may be considered “passive,” since it is applied by a passive airflow resistor, rather than relying on the active application of force (e.g., air). The nasal respiratory devices may be configured to provide resistance to either or both exhalation and inhalation within a specified therapeutic range or ranges for the treatment of apnea, snoring, or other disorders. As used herein, a patient may be any subject, human or non-human, in need of the nasal respiratory (“nasal”) devices described herein or in the incorporated references. These devices may be provided as prescription or non-prescription (“over the counter”) devices.

These patents and patent applications generally describe nasal respiratory devices and methods for treating a variety of medical conditions through the use of such devices. These medical conditions include not are not limited to snoring, sleep apnea (obstructive, central complex and mixed), Cheyne Stokes breathing, UARS, COPD, hypertension, asthma, GERD, heart failure, and other respiratory and sleep conditions. Such nasal respiratory devices typically induce positive end-expiratory pressure (“PEEP”) and/or expiratory positive airway pressure (“EPAP”), and are adapted to be removably secured in communication with a nasal cavity. Similarly, the respiratory devices described herein may include any devices having one or more expiratory resistor valves.

These devices may include a passageway with an opening at a proximal end and an opening at a distal end, a valve (or airflow resistor) in communication with the passageway, and a holdfast to secure the device in communication with a nostril, nasal opening and/or nasal passage. For example, the holdfast may be configured to removably secure the respiratory device within (or over or around) the nasal cavity. The holdfast may be configured to removably secure the respiratory device within (or over or around) the nasal cavity. The airflow resistor (which may be a valve) is typically configured to provide greater resistance during exhalation than during inhalation.

Although general descriptions of these devices have been described both functionally and by example, some specific variations of nasal respiratory devices have not previously been described. Thus, it may be beneficial to improve upon the devices, kits and methods previously described, and particularly to more fully develop certain embodiments of nasal devices and methods of arranging, using, manufacturing, inserting and removing nasal respiratory devices, Described below are specific variations of nasal devices, accessories for nasal devices, methods of using nasal devices and kits including nasal devices.

In addition, it may also be beneficial to adapt these devices to enhance the comfort of a patient using the device, particularly when the device is to be worn while sleeping and applied prior to falling asleep. For example, when wearing nasal devices having an increased resistance to exhalation, some individuals may benefit from a period of adjustment during which they can acclimate to the feel of the nasal device and its effect on their nasal breathing. A subject preparing to wear the device while sleeping may more easily fall asleep once he or she has gotten used to the nasal devices. Thus, it may be beneficial to provide nasal devices and methods of using and making nasal devices that allow control of the onset of resistance to exhalation, suspension of the resistance to exhalation, or delay of the resistance to exhalation (and/or inhalation), allowing time for the subject to acclimate to the feel of the device before the airflow resistance is completely engaged. In particular, it may be beneficial to provide adapters that may be used with any of the previously described (or herein described) devices that allow the subject (or another person) to temporarily decrease the resistance through the device for some period of time (e.g., when falling asleep wearing the device).

Thus, also described herein are nasal devices configured to delay (or suspended) a high baseline resistance that may address the issues raised above are described and illustrated, including methods of using and methods of forming such devices.

### SUMMARY OF THE INVENTION

The present invention relates to layered nasal devices, as well as adapters that may be used with many different types of nasal device (including, but not limited to these layered nasal devices) to enhance comfort by regulating or delaying the onset of resistance through the device. In general, the adapters described herein may be used with any nasal device that provides an increased resistance to exhalation compared to inhalation.

For example described herein delay adapters (which may also be referred to as a delayed resistance adapters or simply adapters) to convert any nasal device that otherwise inhibits exhalation more than inhalation into a delayed resistance nasal device which can be activated to suspend the increase in expiratory resistance.

In some variations, a delay adapter for use with a nasal device to delay the onset of increased resistance to exhalation through the nasal device (wherein the nasal device is otherwise configured to increase the resistance to exhalation more than the resistance to inhalation through the nasal device) includes: a connector configured to connect the adapter to the nasal device; a delay element configured to prevent or reduce the increased resistance to airflow through the nasal device during exhalation for first time period; and a control for activating the delay element.

The connector may be a clip, snap, adhesive, tie, or any other coupling member that secure the adapter to the nasal device. The adapter may be coupled to the nasal device prior to wearing the nasal device, or the adapter may be coupled to a nasal device after it has been placed on the subject. The connector may also serve to align the adapter with the nasal device, and particularly with the airflow resistor region of the nasal device. In some variations, the connector may mate with a complimentary region on the nasal device, and therefore the connector, or a region of the adapter, may be configured to mate with the nasal device and align and/or orient the adapter relative to the nasal device. The connector may be configured to releasably or permanently connect the adapter to a nasal device. For example, in some variations the connector is removable, allowing the adapter to be removed from the nasal device.

The delay element may include any element or structure configured to suspend or interrupt the nasal device to prevent the increase in resistance to expiratory airflow through the nasal device. For example, delay element may be a protrusion (e.g., post, arm, etc) or a passageway (e.g., tube, portal, etc.) that either holds open the airflow resistor during exhalation (and/or during both exhalation and inhalation) to prevent an increase in resistance to exhalation. Non-mechanically interfering member may also act as delay elements, including magnetic elements (e.g., that apply magnetic force or energy to hold open the airflow resistor), adhesive elements (e.g., adhesively holding open the airflow resistor), etc. in some variations the delay element is a bypass that bypasses the airflow resistor and/or the rest of the nasal device, creating or maintaining a relatively low-resistance pathway for airflow during exhalation. In some variations the delay element may both inhibit the airflow resistor and create a bypass pathway for respiration. For example, the delay element may comprise a bypass channel through which air may pass during exhalation.

In general, a control may be used to activate the delay element so that it engages with the nasal device to suspend the increased resistance to exhalation. The control may be a manual control (e.g., switch, button, dial, etc.) or an automatic control (e.g., electronic control, programmable control, etc.). The control may include or be connected to a timer (e.g., timing element) that maintains the activation/engagement of the delay element so that the increased resistance is suspended for a first time period 2 minutes, 5 minutes, 10 minutes, 20 minutes, 30 minutes, 45 minutes, 1 hour, etc.). For example, in some variations the delay element comprises an airflow resistor bypass element configured to extend from the delay adapter and bypass the airflow resistor of the nasal device. In some variations, the delay element may comprise a mechanical delay element configured to hold the airflow resistor at least partially open during exhalation.

In some variations the time period is selectable or programmable. The timer element may be bias or biasing element that engages and/or releases the delay element by taking a first shape and slowly returning to the unbiased shape thereafter.

In some variations the control is engaged automatically when the adapter is applied to the nasal device. For example, the control may be triggered (activated) when the adapter engages the nasal device, thereby suspending the increase in expiratory resistance through the nasal device (relative to the resistance to inhalation). The control and/or timer may be a frangible element that is ‘used up’ during the period that the increase expiratory resistance is suspended, or it may be reusable. For example, the control and/or timer may be a material that melts, sublimates, permanently deforms, etc. when “activated”. For example, the control and/or timer may be a wax element that melts over time when exposed to the increased temperature and/or humidity near or in the subject's nasal passage. In some variations the control and/or timer includes an adhesive material that slowly loses its adhesive properties once activated. Re-usable controls and/or timers may include elastic or shape-memory material that passively (due to material properties) releases the delay element to allow the nasal device to once again inhibit exhalation more than inhalation. Other controls and/or timers may actively move (e.g., by motorized, piezoelectric, solenoid, or other force applicators) or engage the delay element(s) with the nasal device (e.g., the airflow resistor of the nasal device).

Also described herein are delay adapters for use with a nasal device to delay the onset of increased resistance to exhalation through the nasal device (wherein the nasal device is otherwise configured to increase the resistance to exhalation more than the resistance to inhalation through the nasal device), the adapter comprising: a connector configured to connect the adapter to the nasal device; an airflow resistor bypass configured to retractably extend from the delay adapter and bypass the airflow resistor of the nasal device; and a control for activating the airflow resistor bypass to extend from the delay adapter for a first time period.

Also described herein are methods of delaying the onset of an increased resistance to exhalation through a nasal device otherwise configured to increase the resistance to exhalation through the nasal device more than the resistance to inhalation through the nasal device, the method comprising: securing a delay adapter to a nasal device that is configured to inhibit exhalation more than inhalation; and activating the delay adapter to prevent the nasal device from inhibiting exhalation more than inhalation for a first time period.

The step of securing the delay adapter to the nasal device may comprises securing the delay adapter to a passive nasal device worn on a subject's nose. The device adapter may be attached to the nasal device before or after it has been placed on (worn) the subject's nose.

Activating the delay adapter may include triggering a control on the nasal device to engage a delay element with the airflow resistor of the nasal device. For example, activating the delay adapter may comprise preventing the nasal device from inhibiting exhalation more than inhalation for at least 5 minutes, at least 10 minutes, at least 20 minutes, at least 30 minutes, at least 40 minutes, at least 60 minutes, etc.

The adapters described herein may restore the increased resistance to exhalation otherwise provided by the nasal device gradually, quickly or immediately, at or near the end of the first time period. For example, in some variation, the nasal devices may be configured to gradually increase the resistance to exhalation through the nasal device at (or just prior to) the end of the first time period. The adapter may be configured to gradually withdraw the delay element, providing a gradually increasing resistance to exhalation.

Layered nasal device are also described herein. In general, the layered nasal devices illustrated below are typically planar (e.g., flat or flattened) and may be formed by serially layering various regions over or against each other. For example, the layered nasal device may be generally fairly thin (and in some embodiments quite flexible). Such nasal device are typically worn by adhesively applying over one or both nostrils, though other variations that extend at least partially into the nose are also contemplated by this disclosure.

Because the layered nasal devices described herein may be relatively thin and flexible, they may wrinkle, buckle or become miss-aligned with wear and application, particularly if they are improperly applied or handled. Thus, in some variations a nasal device, and particularly a layered nasal device, may include one or more stiffening members or elements that may help ease handling of the nasal device and/or application of the nasal device. In some variations the nasal device includes a stress-distributing element, member, or modification to one or more layers of the nasal device that helps to prevent buckling, wrinkling, or miss-alignment of the nasal device. A stress-distributing element, member, or modification may include a cut-out region that is positioned on or in the layered nasal device to redistribute stress across the nasal device e.g., across the adhesive holdfast layer). Other stress-distributing elements may include stiffening members such as frames, ribs, creases, or the like.

For example, described herein are layered nasal devices that are adapted to be adhesively secured in communication with a subject's nasal cavity, the device having a generally planar body comprising: an airflow resistor configured to inhibit exhalation more than inhalation, wherein the airflow resistor comprises a flap valve layer adjacent to a flap valve layer; an adhesive holdfast layer extending at least partially around the airflow resistor and configured to secure the airflow resistor in communication with the subject's nasal cavity; and one or more stiffening members providing structural support to the adhesive holdfast layer.

In some variations, the one or more stiffing members is configured as a stress-distributing element(s). In some variations the device may include a separate stress-distributing element, member, or modification to a region (e.g., layer) of the nasal device. For example, a stress-distributing element may include one or more cut-out shapes in the stiffening member or other region (e.g., adhesive holdfast layer). The stress-distributing element may be oriented to prevent buckling, wrinkling, or misalignment of the device by damping or re-directing stress in one or more direction as the device is worn. For example, the stress-distributing element may be oriented along an axis (e.g., long axis) of the device, or around the parameter of the device.

In some variations the stiffening member comprises a frame. The stiffening member may extend at least partially around the perimeter of the adhesive holdfast layer, which may help prevent the adhesive holdfast from wrinkling or folding back on itself.

The layered nasal device may be a whole-nose or a single-nose device. For example, the layered nasal device may be a whole-nose nasal device that is configured to communicate with both of a subject's nostrils.

Also described herein are layered nasal devices for inhibiting exhalation more than inhalation, the device comprising: a first skin-contacting adhesive layer; a flap valve layer having a plurality of flaps; a valve limiting layer lying on one side of the flap valve layer and configured to prevent the flaps from opening during exhalation; and a second adhesive layer securing the valve limiting layer to the flap valve layer and the skin-contacting adhesive layer.

As mentioned above, the layered nasal device may include one or more stress-distributing elements, e.g., cut-outs in one or more layers of the nasal device, and the cut-outs may be arranged along the long axis of the nasal device.

Also described herein are layered nasal devices that are generally planar and adapted to be adhesively secured in communication with a subject's nasal cavity, the device comprising: an airflow resistor configured to inhibit exhalation more inhalation, wherein the airflow resistor comprises a flap valve layer lying against a flap valve limiting layer; an adhesive holdfast layer extending at least partially around the airflow resistor and configured to secure the airflow resistor in communication with the subject's nasal cavity; and one or more stiffening members connected to the adhesive holdfast layer supporting the holdfast layer, wherein the stiffening member is configured to include one or more stress-distributing elements.

For example, described herein are layered nasal devices that may be substantially flat and formed of layers (e.g., four or fewer layers). For example, the device may include essentially four layers: a skin-adhesive layer (which may include a liner), an airflow valve layer; a valve-limiting layer, and an adhesive layer between the limiting layer and the airflow resistor layer. The sizes of each layer may be selected or specific.

In general, these nasal devices may be adhesively secured over both of a subject's nostrils, or over one of a subject's nostrils. In sonic variations, the devices may include one or more stiffening members that may further include stress-distributing elements. For example, in some variations, the stress-distributing member includes a cut-out shape to distribute stress in the device.

Also described are delayed resistance adapters. A delayed resistance adapter may regulate the onset of resistance (e.g., expiratory resistance) in a nasal device that includes an airflow resistor to inhibit nasal exhalation more than inhalation. In general, a delayed resistance adapter may include an airflow resistor bypass element and a connector or connector region for securing the adapter to the nasal device, as well as a control for activating the bypass element.

In addition to the disclosure provided herein, Appendix A illustrates further variations and includes values for the sizes and/or thicknesses of these variations. The measurements provided are for illustration purposes only. In some variations, the measurements may each be +/− a percentage of the indicated value (e.g., +/−5%, 10%, 15%, 20%, 25%, 30%, etc.).

Further, the ration of these sizes/measurements may be maintained (e.g., the relative lengths of the various layers) may be maintained.

### BRIEF DESCRIPTION OF THE DRAWINGS

FIG. 1 shows a nasal device including a rim.

FIG. 2 is a nasal device including a stress-distributing element.

FIG. 3 is a top view of a streamlined layered nasal device such as the example shown in FIG. 2.

FIG. 4 is a bottom view of the nasal device shown in FIG. 3.

FIG. 5 is an exploded view showing the various components of the layered nasal device of FIGS. 3 and 4.

FIGS. 6A-6D illustrate various embodiments of layered nasal devices. FIG. 6A shows a layered nasal device having 9 layers. FIGS. 6B-6D illustrates variations of layered nasal device having four layers (the bottom layer is an optional liner).

FIG. 7A shows an exploded view of one variation of a layered nasal device similar to the device shown in FIG. 6C show. FIG. 7B shows a collapsed (top) view of the device shown in FIG. 7A.

FIG. 7C shows a bottom view of the nasal device of FIGS. 7A and 7B. FIG. 7D illustrates exemplary dimensions of one variation of a nasal device such as the one shown in FIG. 7A (exemplary dimensions are also shown in FIG. 7C).

FIGS. 7E and 7F show schematic illustrations of a flap valve layer (FIG. 7C) and an adhesive holdfast layer (FIG. 7F) for a device such as the one shown in FIGS. 7A-7D. FIGS. 7G and 7H show schematic illustrations of a valve limiter layer (FIG. 7G) and double-adhesive connecting layer (FIG. 7H) for the device such as the one shown in FIGS. 7A-7D. As mentioned above, the dimensions shown are merely illustrative and should not be limiting.

FIG. 8A shows a top view (the patient-contacting side) of a nasal device, and FIG. 8B shows a bottom view of the same nasal device.

FIG. 8C shows a side perspective view of a delayed resistance adapter connected to one variation of a nasal device.

FIG. 9A shows one variation of a delayed resistance adapter and FIG. 9B shows another variation of a delayed resistance adapter.

FIGS. 10A and 10B are variations of a delayed resistance adapter to be connected to a nasal device.

### DETAILED DESCRIPTION OF THE INVENTION

### Part I: Layered Nasal Devices

As mentioned above, a layered nasal device may include one or more stress-relieving elements or features. Stress-relieving features may be selected so that they prevent stress (e.g., mechanical stress) from de-laminating, buckling, wrinkling, or otherwise disrupting the shape and/or activity of the nasal device. In general, a layered nasal device may be applied over one or both of a subject's nostrils, and secured over (or partially within) the subject's nose to regulate airflow through the nose. The nasal device may include an adhesive holdfast that secures (or helps secure) the nasal device in position. The nasal device may stressed, for example, if the nasal device is secured under tension, or if the nose or nostrils are moved by the subject or acted upon e.g., by lying against the face when sleeping).

A stress-relieving feature may be incorporated into the nasal device to prevent de-laminating, buckling, wrinkling, or miss-alignment of the nasal device, or portions of the nasal device including the airflow resistor. Examples of stress-relieving features may include cut-outs, flexible regions, framed or partially framed regions; each of these features may be arranged or integrated into the nasal device so that stress in one or more direction is re-directed or dampened.

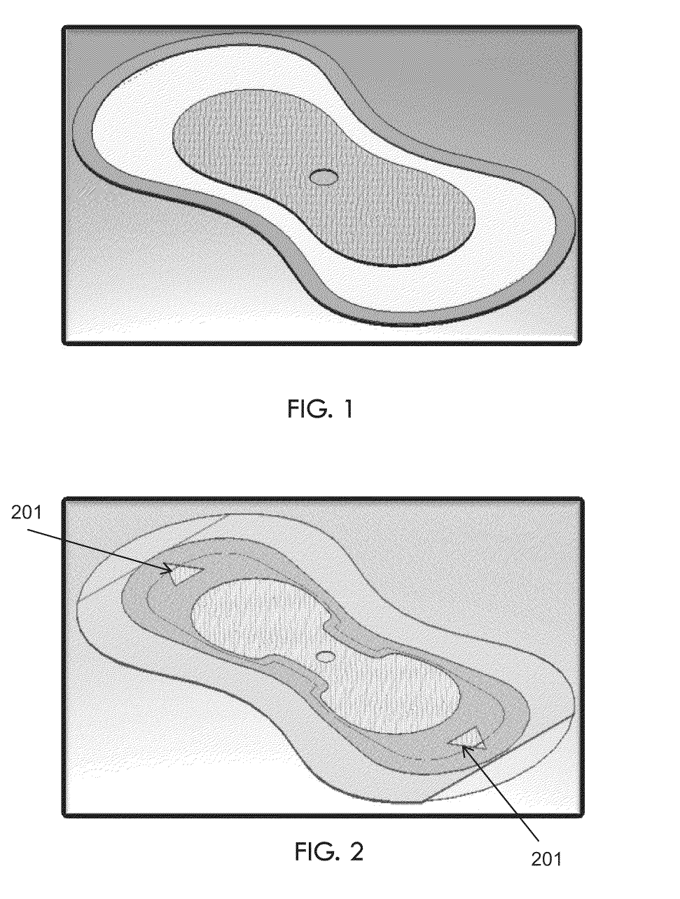

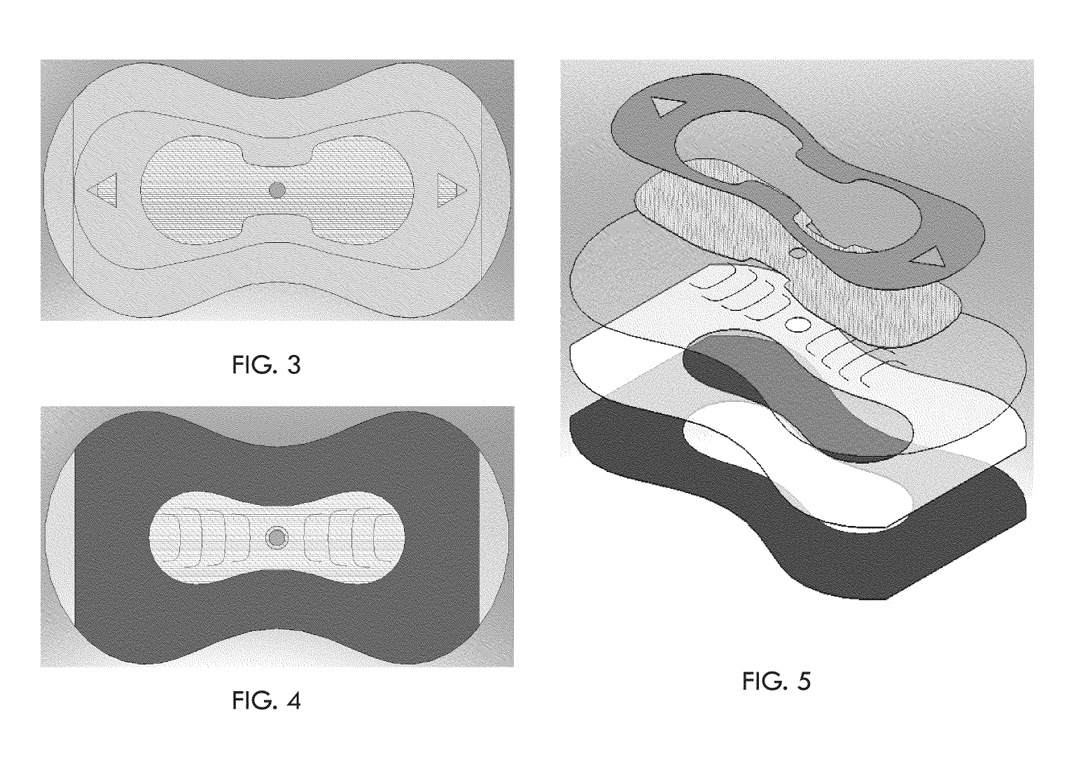

For example, FIG. 1 shows one variation of a nasal device. This example is a layered nasal device including an outer-perimeter rim that may help provide stiffness to the nasal device (e.g., preventing the outer edges from curling during handling and prior to application). FIG. 2 shows another variation of a nasal device including stress-relieving features arranged in the device to distribute the stress across the long axis of the device. In FIG. 2, the two triangular cut-out regions 201 of the device are arranged (symmetrically) along the long-axis of the nasal device. These cut-outs are formed in the adhesive that secures the valve-limiting layer (mesh) to the valve-forming layer, as illustrated in FIG. 5. FIGS. 3 and 4 illustrate top and bottom views, respectively, of this example,

FIGS. 2-5 also show one variation of a layered nasal device including four layers. For the purposes of this application, the removable backing material is not considered a device layer, as it is removed at the time of applying the nasal device. The tour-layer variation shown in these figures includes a bottom skin-adhesive layer. Both sides of this adhesive layer are adhesive, so that the device can be applied to the subject's nose or nostrils. The adhesive layer may be backed by a peel-away backing layer (as mentioned). In some variations the adhesive is activated by moisture, solvent, or otherwise, and a separate backing layer is not necessary. The adhesive layer may be connected directly to the valve-forming layer, sealing around the perimeter of the flaps (valves). As shown here, the valve layer may include a plurality of flaps cut through the layer. A valve-limiting layer e.g., mesh) may directly abut one side of valve-forming (flap) layer, and a second adhesive (one—sided in these examples) may be around the perimeter of the valve-limiting layer to hold it in place against the valve-forming layer.

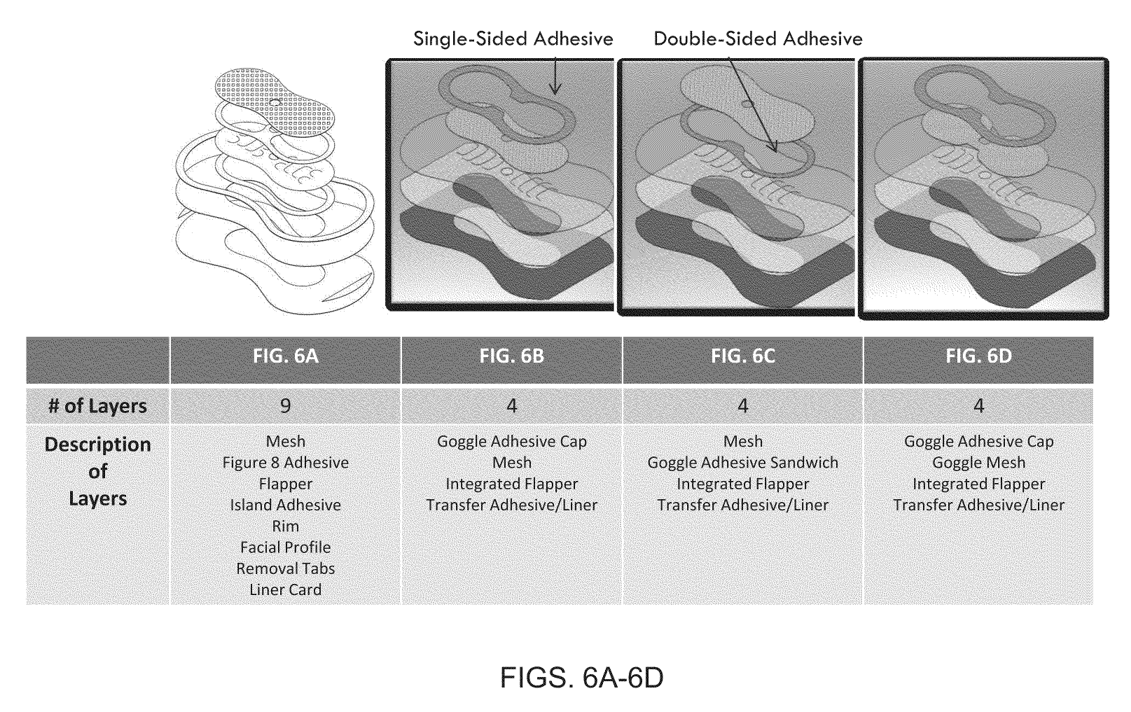

FIGS. 6A-6D illustrate different variations of layered devices. For example, in FIG. 6A, a layered nasal device is shown comprising 9 components. These components include a mesh (valve-limiting) layer, a first adhesive layer (“island adhesive layer”), a valve-forming (flapper) layer, a second adhesive layer, a rim layer, a skin-adhesive layer, two removal tabs and a liner card that is not the original adhesive liner. Since each of these components may be applied as separate layers, this may be referred to as a nine-layer embodiment.

FIGS. 6B-6D illustrate variations having four layers. These four layers may be arranged in a different order, as indicated in the table below the figures. For example, FIG. 6B shows a variation including a single-sided adhesive “cap” layer followed by a valve-limiting (mesh) layer, a valve-forming (flap) layer, and then a skin-adhesive layer (“transfer adhesive and liner” layer). Alternatively, FIG. 6C shows a variation using a double-sided adhesive that is ordered by aligning an outer valve-limiting (mesh) layer atop a double-sided adhesive (“sandwich” layer), then a valve-forming layer and a skin-adhesive layer (“transfer adhesive and liner” layer). FIG. 6D shows a variation similar to that of FIG. 6B, however the valve-limiting layer (mesh layer) is trimmed near the waist (middle) region. This cut-out region is complimentary to the regions projecting from the adhesive waist region that extends towards the opening in the center of the device (leak pathway). The (optional) adhesive liner layer in FIGS. 6B-6D is the liner that is supplied with the adhesive, and so is not considered a layer.

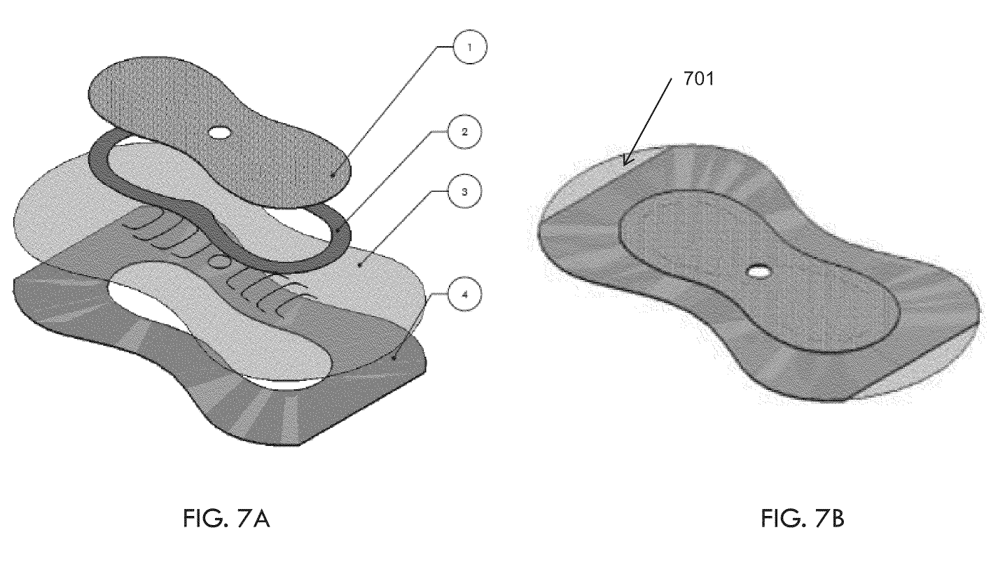

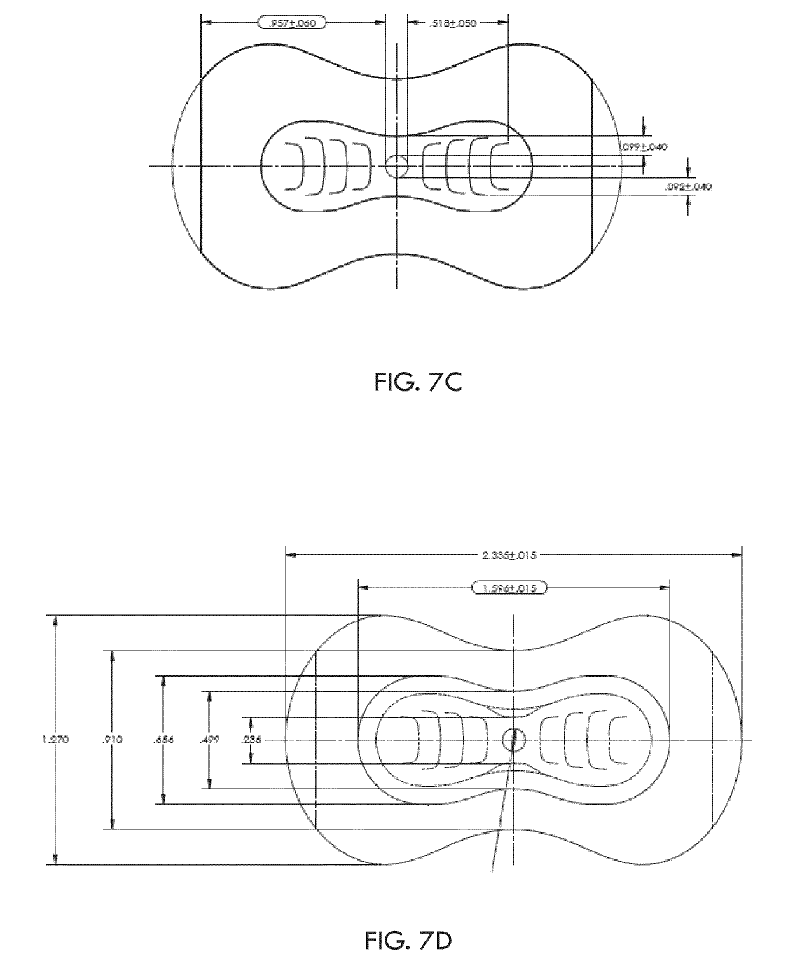

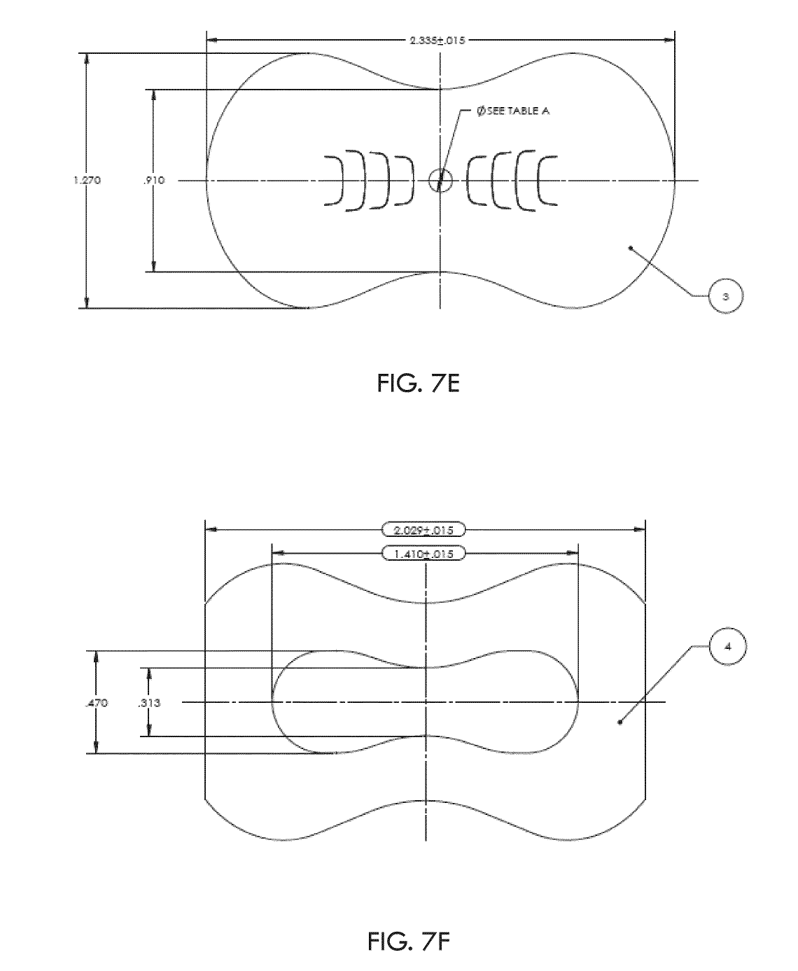

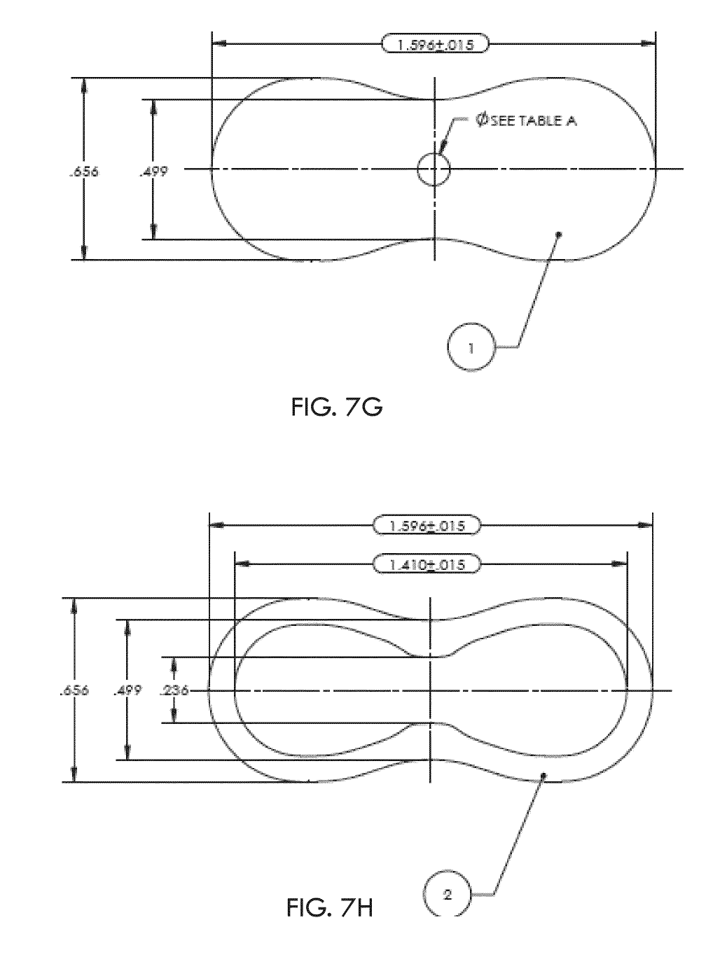

FIGS. 7A-7H illustrate another variation of a layered nasal device. In these figures, various dimensions of the layers are shown for illustrative purposes only. All or some of these dimensions may be modified (e.g., increased, decreased, etc.) alone or in combination; in some variations these dimensions may be scaled to increase or decrease the sizes of exemplary devices shown. For example, FIG. 7A shows an exploded view of one variation of a nasal device, similar to the variation shown in FIG. 6C. FIG. 7B shows an assembled view of this nasal device (seen from the outward-facing surface or bottom), also including a liner backing that may be removed to expose the adhesive layer 4. In this variation the adhesive layer is double-sided adhesive, to secure both the adjacent flap valve layer 3 and the skin-contacting slide (which may be removably covered by a liner 701). FIG. 7C shows top view of the assembled device, and FIG. 7D a view of the assembled device with the layers made transparent. In FIGS. 7C to 7H, exemplary sizes have been provided. The four layers forming the assembled device is illustrated in greater detail in FIGS. 7E-7H; FIG. 7E shows one variation of a flap valve layer 3 and FIG. 7F shows one variation of an adhesive holdfast layer 4. FIG. 7G shows one variation of a valve limiting layer 1, while FIG. 7H shows one variation of an annular adhesive 2 to secure the valve limiting layer 1 adjacent to the flap valve layer 3.

In general, the mesh region may be configured to be sufficiently low resistance (e.g., allowing a very low resistance to inhalation, while supporting the valve flaps during exhalation to increase the resistance. For example, the mesh may have a large opening size, or it may have a large open area (or a larger “pore” or “window” size), while still supporting the flaps of the valve layer from opening substantially during exhalation.

As mentioned briefly above, the non-skin contacting adhesive layer may include “goggle” regions near the waist region. These regions extend partially (but not completely) across the device, near the opening (the leak pathway). Although the adhesive may extend completely across the device, forming an “island” and have a hole through it corresponding to the leak pathway, in some variations it may be beneficial to extend only partially across this midline, avoiding the necessity for punching a hole through the adhesive, without compromising the support of this region. Removal of the adhesive from the center region completely may compromise the connection between the two layers (i.e., the limiting layer and the valve layer), and may allow “billowing” of the center of the device, potentially altering the predictability of the airflow resistances. Extending the adhesive partially across this region as shown may help prevent this.

In some variations, the leak pathway is formed around the cuts forming the flaps, instead of (or in addition to) a dedicated leak pathway such as the central opening. An increase in resistance loss around the flappers may be used to titrate the resistance to inhalation (and/or exhalation).

### Part II: Delayed Resistance Adapters

As mentioned above, described herein are adapters to regulate the onset of resistance (e.g., expiratory resistance) in a nasal device that includes an airflow resistor to inhibit nasal exhalation more than inhalation. Such adapters may allow the resistance to exhalation in the nasal device to be suspended or delayed. Suspension or delay of onset of the increase in resistance to exhalation may permit a subject to acclimate to wearing a nasal device that inhibits exhalation more than inhalation, particularly when the device is applied prior to sleeping.

In general, a delayed resistance adapter includes an airflow resistor bypass element and a connector or connector region for securing the adapter to the nasal device, as well as a control for activating the bypass element. A resistor bypass element typically bypasses or disables the airflow resistor (e.g., flap valve) so that it is bypassed or held in an open, or non-resistive, configuration.

The delayed resistance adapters described herein may be used (or configured for use) with any appropriate nasal device, particularly those described in the patents and patent applications incorporated by reference above. For example, the delayed resistance adapters may be used with a layered nasal device as described above, or a nasal device including a separate rim body region. A delayed resistance adapter may also be referred to as airflow resistor bypass adapter.

The delayed resistance adapters described herein may incorporate and be similar to any of the airflow resistor bypass mechanisms and elements illustrated in co-pending patent application U.S. patent application Ser. No. 12/329,895, filed on Dec. 8, 2008, titled “DELAYED RESISTANCE NASAL DEVICES AND METHODS OF USE,” and incorporated by reference above; these devices typically also include a connector or connector region for securing the adapter to a nasal device.

A delayed resistance bypass adapter may be configured to suspend the resistance (e.g., expiratory resistance) when activated either for a limited (e.g., predetermined) amount of time or indefinitely. The bypass adapter may be turned “on” by operation of the control button, lever, etc.). In some variations the bypass adapter may be turned “off” by the same (or a separate) control, or it may be automatically turned off. In some variations the device may be gradually turned “off”; for example, the adapter may gradually increase the resistance to exhalation through the nasal device.

The delayed resistance adapters described herein may be permanently or removably attached to a nasal device. Thus, in some variations, the delayed resistance adapters may be re-used on one or more different nasal devices. For example, the connector may be a glue or other adhesive that may permanently bond or connect the adapter to a nasal device. In some variation the delayed resistance adapter is a snap, friction fit, magnetic connection, latch and hook, or other re-usable connectors that mate with a connector or connector region of the nasal device.

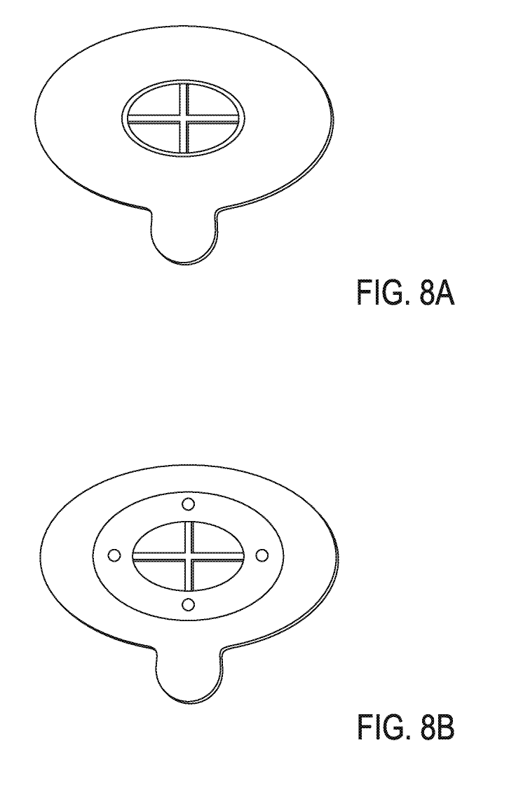

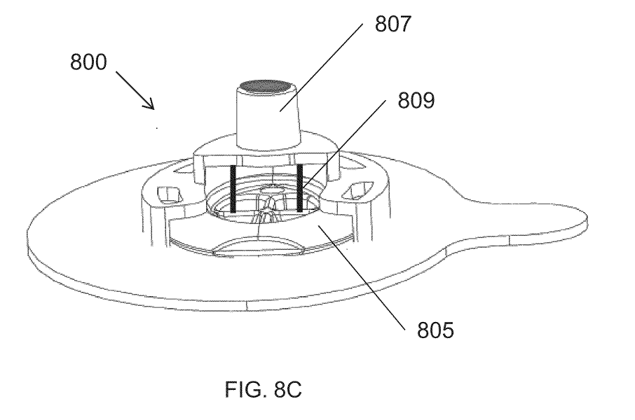

FIGS. 8A and 8b show top and bottom views, respectively, of one variation of a nasal device. In this example, the nasal device is an adhesive nasal device that can be secured over a subject's nostril. The top side (FIG. 8A) is the skin-contacting side, while the bottom side (FIG. 8B) faces away from the subject wearing the nasal device. The nasal device includes a rim body region (visible in FIG. 8B), or it may be a layered nasal device that does not include an explicit rim body region.

FIG. 8C shows one variation of a delayed resistance adapter 800 secured to the bottom of a nasal device such as the one shown in FIGS. 8A and 8B. In this figures, the delayed resistance adapter 800 is friction tit onto the rim body region 805 of the nasal device by snapping it into position. The delayed resistance adapter includes a delay bypass mechanism that includes an airflow resistor bypass element 809 that can extend from the delayed resistance adapter 800 to interfere with the airflow resistor. In FIG. 8C two delay bypass mechanisms 809 are visible extending from the delayed resistance adapter. In this example, the delay bypass mechanism(s) include one or more posts that extend into the airflow resistor, holding it open and preventing it from closing and increasing the airflow resistance during exhalation. This example also includes a control 807 that can be used to activate the delay bypass mechanisms, causing them to extend into the flap valves forming the airflow resistor and prevent one or more of the flap valves from closing during exhalation. The control may be operatively connected to a timing mechanism (e.g., electric timing mechanism, mechanical timing mechanism, etc.) that either holds the delay bypass mechanisms in position (preventing closing of the airflow resistor) for some time period prior to retracting them and allowing the application of respiratory resistance. The delayed resistance adapter may be configured to gradually release the delay bypass mechanism, or to suddenly release it.

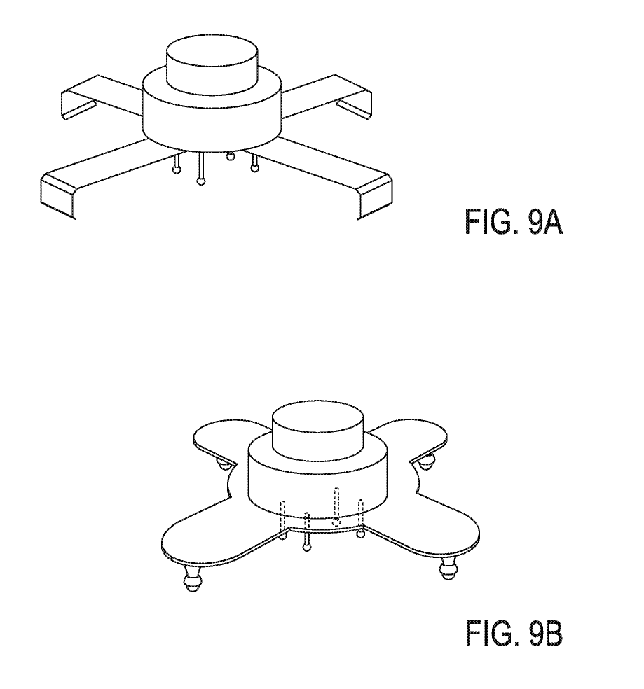

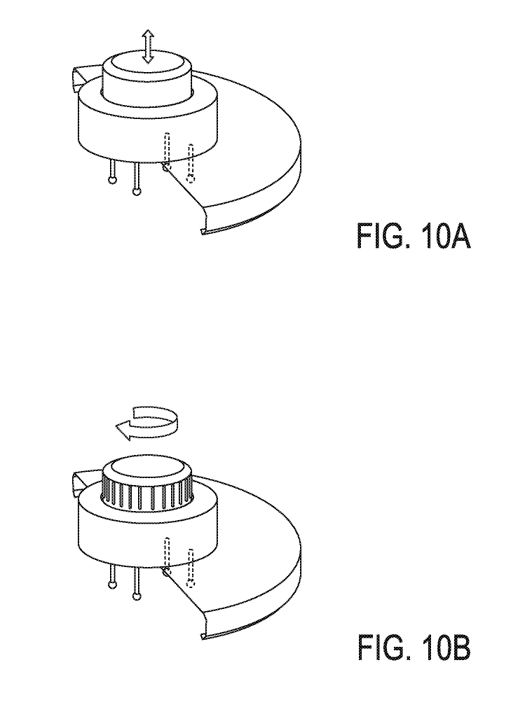

FIGS. 9A-10B show other variations of delayed resistance adapters. For example, FIG. 9A shows one variation of a delayed resistance adapter that includes a connector region having four arms that can grasp a portion of the nasal device (e.g., the rim body region) similar to the device shown in FIG. 8A-8C. The variation shown in FIG. 9B is similar, but includes coupling members 903 at the distal ends of the four arms. These coupling members may be configured to mate with and engage attachment sites on the nasal device.

In all of the variations of adapters illustrated, the bottom of the adapter may be separated from the nasal device when the adapter is attached to permit the passage of air through the device substantially uninhibited by the adapter.

FIGS. 10A and 10B illustrate different variations of delayed resistance adapters having friction-fit connectors similar to the connector shown in FIG. 8C. In FIG. 10A, the adapter includes a button-type control similar to those already illustrated in FIGS. 8C to 9B. This control may be activated by pushing the button, for example, FIG. 10B shows another variation in which the adapter control comprises a dial that may be rotated (or in some cases a button that may be pushed). Thus, the control may be activated to set a duration for the delay.

As mentioned above, some patients have difficulty getting used to an expiratory resistance device due to difficulty in falling asleep at the beginning of the night or falling back asleep after they wake up in the middle of the night. Thus, allowing the expiratory resistance to be deactivated temporarily and then to automatically re-activate after a period of time (e.g. 15 minutes, 20 min, 30 min, etc.) may allow the patient to fall asleep without any (or with nominal) resistance but have the device turn on when the patient is asleep. Ideally this feature may be reactivated several times throughout the night.

Any of the adapters described herein may be separate, reusable “snooze feature” type devices (adapters). As mentioned, such adapters may deactivate the resistance mechanism of a nasal device having expiratory resistance. These adapters may therefore also be referred to as “snooze” devices or adapters, and can be attached to the nasal device in a way that allows the user to put it on at night and remove it in the morning. The resistance device could be thrown away but the snooze device could be saved and reused for a number of nights. This would significantly reduce or eliminate the cost constraints on the device since the cost could be spread over a number of nights and every resistance device would not need to individually include this feature when it may not be needed every night. Thus a snooze type adapter device could attach to the resistance device in several ways and may not even need to be attached but just positioned in proximity. For example, the adapters described herein may include a connector configured as a snap mechanism. The advantage of this approach is that it would make sure that the snooze device was always positioned relative to the nasal device in the same way each time; it is also relatively inexpensive to manufacture. There are other snap or adhesive configurations that could also be developed to hold the snooze device in place. As described above, the deactivation mechanism could be a post or posts that push the flapper valve on the device open so that they do not close during expiration. This could engage with one or multiple valves in the device. The snooze feature could also include timing mechanisms such as springs, foams, or other mechanisms that mechanically displace over time. Additionally, this concept could include more elaborate electronic mechanisms such as battery operated mechanisms that power the arm and this could even include multiple settings for time delay before reactivation. These devices could also include a mechanism to slowly increase the expiratory resistance in addition to a quick on/off. This could be done by either re-enabling multiple valves in a time delayed step manner or by reducing the degree of displacement of the valves over time.

While the devices (and methods for using them) have been described in some detail here by way of illustration and example, such illustration and example is for purposes of clarity of understanding only. It will be readily apparent to those of ordinary skill in the art in light of the teachings herein that certain changes and modifications may be made thereto without departing from the spirit and scope of the invention.

What is claimed is:

1. A delay adapter for use with a nasal device to delay the onset of increased resistance to exhalation through the nasal device, wherein the nasal device is otherwise configured to increase the resistance to exhalation more than the resistance to inhalation through the nasal device, the adapter comprising:
a connector configured to connect the adapter to the nasal device;
a delay element configured to prevent or reduce the increased resistance to airflow through the nasal device during exhalation for a first time period; and
a control for activating the delay element.

2. The adapter of claim 1, wherein the delay element comprises an airflow resistor bypass element configured to extend from the delay adapter and bypass the airflow resistor of the nasal device.

3. The device of claim 1, wherein the delay element comprises a mechanical delay element configured to hold the airflow resistor at least partially open during exhalation.

4. The device of claim 1, wherein the delay element comprises a post.

5. The device of claim 1, wherein the delay element comprises a bypass channel through which air may pass during exhalation.

6. The adapter of claim 1, wherein the connector is configured to releasably connect the adapter to a nasal device.

7. The adapter of claim 1, further comprising a timer that is triggered by the control to determine the first time period.

8. The adapter of claim 7, wherein the timer comprises a mechanical timer.

9. The adapter of claim 1, wherein the control comprises a button.

10. A delay adapter for use with a nasal device to delay the onset of increased resistance to exhalation through the nasal device, wherein the nasal device is otherwise configured to increase the resistance to exhalation more than the resistance to inhalation through the nasal device, the adapter comprising:
a connector configured to connect the adapter to the nasal device;
an airflow resistor bypass configured to retractably extend from the delay adapter and bypass the airflow resistor of the nasal device; and
a control for activating the airflow resistor bypass to extend from the delay adapter for a first time period.

11. The adapter of claim 10, wherein the connector is configured to releasably connect the adapter to a nasal device.

12. The adapter of claim 10, wherein the airflow resistor bypass comprises a post configured to extend from the adapter and into a flap valve of the nasal device.

13. The adapter of claim 10, further comprising a timer coupled to the control, wherein the tinier mechanism triggers retraction of the airflow resistor bypass after the first time period.

14. The adapter of claim 13, wherein the timer is a mechanical timer.

15. The adapter of claim 10, wherein the control is a button.

16. The adapter of claim 10, wherein the control is a dial.

17. A method of delaying the onset of an increased resistance to exhalation through a nasal device otherwise configured to increase the resistance to exhalation through the nasal device more than the resistance to inhalation through the nasal device, the method comprising:
securing a delay adapter to a nasal device that is configured to inhibit exhalation more than inhalation; and
activating the delay adapter to prevent the nasal device from inhibiting exhalation more than inhalation for a first time period.

18. The method of claim 17, wherein securing the delay adapter to the nasal device comprises securing the delay adapter to a passive nasal device worn on a subject's nose.

19. The method of claim 17, wherein activating the delay adapter comprises triggering a control on the nasal device to engage a delay element with the airflow resistor of the nasal device.

20. The method of claim 17, wherein activating the delay adapter comprises preventing the nasal device from inhibiting exhalation more than inhalation for at least 5 minutes.

21. A layered nasal device that is adapted to be adhesively secured in communication with a subject's nasal cavity, the device having a generally planar body comprising:
an airflow resistor configured to inhibit exhalation more than inhalation, wherein the airflow resistor comprises a flap valve layer adjacent to a flap valve limiting layer;
an adhesive holdfast layer extending at least partially around the airflow resistor and configured to secure the airflow resistor in communication with the subject's nasal cavity; and
one or more stiffening members providing structural support to the adhesive holdfast layer.

22. The device of claim 21 further comprising one or more stress-distributing elements.

23. The device of claim 22, wherein the stress-distributing element comprises cut-out shapes in the stiffening member.

24. The device of claim 22, wherein the stress-distributing element is oriented to prevent buckling, wrinkling or misalignment of the device by damping or re-directing stress in one or more direction as the device is worn.

25. The device of claim 21, wherein the stiffening member comprises a frame.

26. The device of claim 21, wherein the stiffening member extends at least partially around the perimeter of the adhesive holdfast layer.

27. The device of claim 21, wherein the layered nasal device is configured to communicate with both of a subject's nostrils.

28. A layered nasal device for inhibiting exhalation more than inhalation, the device comprising:
a first skin-contacting adhesive layer;
a flap valve layer having a plurality of flaps;
a valve limiting layer lying on one side of the flap valve layer and configured to prevent the flaps from opening during exhalation; and
a second adhesive layer securing the valve limiting layer to the flap valve layer and the skin-contacting adhesive layer.

29. The device of claim 28, further comprising one or more stress-distributing elements.

30. The device of claim 28, further comprising one or more stress-distributing elements comprising cut-outs in one or more layers of the nasal device, wherein the cut-outs are arranged along the long axis of the nasal device.

31. A layered nasal device that is generally planar and adapted to be adhesively secured in communication with a subject's nasal cavity, the device comprising:
an airflow resistor configured to inhibit exhalation more inhalation, wherein the airflow resistor comprises a flap valve layer lying against a flap valve limiting layer;
an adhesive holdfast layer extending at least partially around the airflow resistor and configured to secure the airflow resistor in communication with the subject's nasal cavity; and
one or more stiffening members connected to the adhesive holdfast layer supporting the holdfast layer, wherein the stiffening member is configured to include one or more stress-distributing elements.
